(19) United States
(12) Patent Application Publication
(10) Pub. No.: US 2006/0034837 A1
(43) Pub. Date: Feb. 16, 2006

(54) Method and agents for the prevention, inhibition and therapy of cancers

(75) Inventor: Andreas Bergmann, Berlin (DE)

Correspondence Address: MAYER, BROWN, ROWE & MAW LLP, 1909 K STREET, N.W., WASHINGTON, DC, 20006, US

(73) Assignee: B.R.A.H.M.S Aktiengesellschaft, Henningsdorf (DE)

(21) Appl. No.: 10/512,934

(22) PCT Filed: Apr. 4, 2003
(86) PCT No.: PCT/EP03/03553

§ 371 Date: Jun. 3, 2005

(30) Foreign Application Priority Data
May 2, 2002 (EP) 2009882.8

(51) Int. Cl. A61K 31/739; A61K 39/395

(52) U.S. Cl. 424/140.1; 514/54

(57) ABSTRACT

Disclosed are uses of Gangliosides G_{M1} and/or asialo-G_{M1} substances simulating the carbohydrate portion of said gangliosides with regard to bonding to anti-G_{M1} antibodies and/or anti-AD_{M1} antibodies for producing agents which bind or block anti-G_{M1} antibodies and/or anti-AG_{M1} antibodies which bond to natural killer cells (NKC) or for blocking antigen-presenting cells and producing a T-cell anergy, and for producing an affinity material for the extracorporeal removal of anti-G_{M1} antibodies and/or anti-AG_{M1} antibodies I order to prevent, inhibit, and treat malignant cancers.

The present invention relates to novel therapeutic methods for cancer prevention or cancer therapy and the agents which can be used in such therapeutic methods. It is based on the detection, for the first time, of the occurrence of antibodies (autoantibodies) having properties of inhibiting the so-called “natural killer cells” (NK cells) in substantially all patients who suffer from cancer diseases of a very wider range of tissues and organs, and on the role which these antibodies play in the development of cancer, owing to their properties of inhibiting NK cells.

Terms such as “neoplasm”, “cancer” or “carcinoma” are used in the context of the present Application substantially as synonyms for tumour diseases, in particular malignant tumour diseases. In a country such as Germany, more than 300,000 men and women become ill with malignant neoplasms, i.e. cancer, every year, the number of new cancer cases diagnosed annually and the mortality being considerable (cf. for example http://www.medicine-worldwide.de/krankheiten/krebs/xxx.html, where /xxx should be replaced, for example, by /allgemeines; /lungenkrebs; /brustkrebs; /prostatakrebs; /darmkrebs; /leukaemie; /corpuskarzinom). Malignant neoplasms can occur in virtually any tissue or organ and, depending on the organ affected, a distinction is made between numerous cancer diseases which may differ considerably from one another with respect to their statistical frequency, prognosis and treatability.

In this context, it is known that there is an increased risk of cancer for certain persons with a particular disposition to develop cancer diseases, possibly in combination with exposure during work, a medication or previous disease which with high statistical frequency shows malignant development, i.e. is transformed into a cancer. Being able to detect such persons at particular risk and then being able to protect them by enhanced monitoring and/or suitable preventive measures is an important aim of modern cancer research. A research approach which has this aim relates to genome research. Another, immunological approach to the recognition of a particular risk of cancer and to the resulting cancer prevention, cancer inhibition and cancer therapy forms the subject matter of the present Application.

The above-mentioned previous diseases, which are known to degenerate with high probability later in their course into malignant diseases (intestinal cancer), include, for example, the so-called chronic inflammatory intestinal diseases which are known by the names Crohn's disease (synonym: Enteritis regionalis) and ulcerative colitis (synonym: Colitis gravis) and Colitis indeterminata, a mixed form of the two above-mentioned diseases. These are of particular importance for the economy and internal medicine, owing to the high associated morbidity (disease rate) and mortality (death rate). Characteristic symptoms are abdominal pain, diarrhoeas and, where the small intestine is affected, possibly malabsorption. Intestinal stenoses, internal and external fistulas and abscesses occur as complications in Crohn's disease, whereas in some cases severe blood losses and a significantly increased risk of the occurrence of carcinomas of the large intestine (about 40% in the 25 year course of the disease) are observed in the case of ulcerative colitis. If it were possible, for example, to detect persons particularly at risk within the group of patients suffering from chronic inflammatory intestinal diseases and to protect them therapeutically to a greater extent, this would constitute considerable medical progress.

Recent years have seen the development of a large number of therapies which have brought considerable progress with respect to the curability of numerous forms of cancer diseases. In all cases, however, early diagnosis of cancer, i.e. diagnosis of neoplasms at a time when no clinical symptoms or no significant clinical symptoms have as yet occurred, is very particularly important.

For the detection of neoplasms as early as possible in clinical diagnosis, so-called “tumour markers” are determined in biological samples, in particular blood samples, of patients investigated. Tumour markers are substances which are either formed directly by malignant tumour cells or which form by virtue of the fact that tumour cells induce the synthesis of the respective marker in non-tumour cells. If tumour markers are localized in increased concentration in biological fluid samples (humoral tumour markers) or in tissue (cellular tumour markers), they make it possible to draw conclusions about the presence, the course and the prognosis of a tumour disease. The tumour markers currently introduced into clinical diagnostic practice may be oncofoetal antigens, carbohydrate epitopes detectable with monoclonal antibodies, enzymes, isoenzymes, oncogenic products and receptors. An overview of the tumour markers currently used in clinical diagnosis is to be found, for example, in: Lothar Thomas (editor), Labor und Diagnose [Laboratory and Diagnosis], 5th extended edition, Chapter 34: Tumormarker [Tumour markers], pages 956-1019.

In spite of the progress in cancer therapy which has been discussed, there is still a considerable need for novel improved cancer therapies and cancer prevention measures or novel agents effective in the stated context, in particular those which attack at a central point of the origin of cancer and development.

The present invention is based on the surprising finding that a certain antibody or autoantibody type which is known per se in other contexts and has the properties of inhibiting NK cells is found at diagnostically significantly increased levels in all tested malignant neoplasms (cancer types) in biological samples, in particular patients' sera, as far as it has been possible to check experimentally at present, whereas the same antibody is not detectable, or detectable only in substantially smaller amounts, in healthy normal persons. Owing to its properties of inhibiting NK cells, this cell type is causally linked to a cancer process.

By determining these antibodies, it is possible, according to the present invention, to detect with high reliability the presence of malignant neoplasms, for example in patients or persons who are undergoing a routine examination or take part in a serial investigation, without a significant number of healthy normal persons being determined as false positive. If the certain antibody also occurs in increased concentrations in the case of some other special diseases, it is as a rule possible, without major difficulties, to correctly interpret the results of measurements while excluding such a disease, on the basis of additional clinical findings.

If the antibodies inhibiting NK cells are detected in a patient, they become a therapeutic target. With the use of measures known in principle per se in some cases or agents according to the invention for fighting, binding and rendering harmless pathogenic (auto)antibodies and/or of measures with the aim of specifically influencing the antibody formation by the immune system, they can then become the target of preventive, inhibitory and/or therapeutic measures.

The present invention starts from the surprising findings from measurements of sera of normal persons and cancer patients by the Applicant with the aid of a ligand binding assay with high sensitivity for antibodies binding to the gangliosides AG_{M1} and G_{M1}, namely that, substantially in all measured cancer sera, antibodies were found which bind to ganglioside structures and to antigen structures simulating ganglioside structures, and especially to asialo-G_{M1}-binding antibodies (anti-AG_{M1} antibodies) and/or antibodies cross-reacting therewith and binding to monosialo-G_{M1} antibodies (anti-G_{M1} antibodies) of the IgG and/or IgA type. The presence of antibodies which bind to asialo-G_{M1} and thus have properties of inhibiting NK cells, and/or their amount significantly increased compared with normal persons, can thus be directly correlated with the presence of a neoplasm and/or an increased risk to the patient from malignant neoplasms.

The occurrence of antibodies which inhibit NK cells can be causally linked to the origin and spread of a cancer process, it being possible to derive therefrom uses and agents which are accessible to patent protection and which are involved in the inhibition/elimination of such antibodies and for which protection is desired in Patent Claims 1 to 8.

The significance of the diagnostic determination of anti-ganglioside antibodies or autoantibodies for the diagnosis and for the therapy of cancer genesis or cancer development is explained in more detail in the second part of this Application.

The present invention is an unexpected result of intensive researches by the Applicant in the area of the clinical diagnosis of autoimmune diseases. They started from the knowledge that certain antiganglioside antibodies are also among the antibodies which are discussed in the literature in relation to autoimmune diseases, in particular nerve-damaging, neuropathic autoimmune diseases.

Gangliosides are glycolipids which are constituents of the extracellular side of the plasma membrane of animal cells and as such also occur in nerve tissue. They contain several monosaccharide units per mole but have no phosphorus content and are assigned to the sphingolipids. Compared with proteins, they tend to be low molecular weight biomolecules. The gangliosides to which the antibodies discussed in the context of the present invention bind are the monosialo-ganglioside referred to generally as G_{M1} and in particular the associated “asialo” compound AG_{M1}. G_{M1} has a polysaccharide chain of 4 sugar monomer units which comprise two D-galactose units, one N-acetylgalactosamine unit and one D-glucose unit, the latter being bound to a ceramide moiety. In the ganglioside G_{M1}, an N-acetylneuraminic acid radical (NANA; sialic acid or o-sialinic acid radical; “monosialo” radical), which is missing in the sialinic acid-free asialo-G_{M1} (AG_{M1}), is bound to the D-galactose unit arranged inside the polysaccharide chain.

Said gangliosides and related compounds are associated with numerous important biological functions of the human body, including, for example, axonal growth and neuronal differentiation, receptor functions and participations in various immune reactions of the body and in signal transduction and cell-cell recognition.

It has long been known that antibodies or autoantibodies which bind to the ganglioside G_{M1}, in particular its carbohydrate structures, and to carbohydrate structures of other molecules which resemble these (“simulate these”) can occur in the human body. The physiological role of such antibodies and their possible importance for clinical diagnosis are the subject of numerous scientific investigations. The findings for anti-G_{M1} antibodies have not yet been correlated in this context with properties known per se for antibodies binding to asialo-G_{M1}.

By far the predominant part of all published papers are concerned with the role and the diagnostic significance of anti-ganglioside antibodies in neuropathies, for example in immunomediated motor neuropathies, such as Guillain-Barré syndrome (radiculoneuritis, polyradiculitis) and the related (Miller-)Fisher syndrome. An increased occurrence of anti-G_{M1} autoantibodies in some patients was also reported in association with Alzheimer's disease. Furthermore, they were found in individual HIV patients. Individual experiments for their determination in association with certain cancer types were also reported, but only few informative results or results with a low sensitivity were obtained. In this context, reference is made to the discussion further below.

In order to avoid unjustifiably narrow and restrictive interpretations of the terms used in the present Application and the associated claims, some of the most important terms are to be defined in particular below for the purposes of the present Application:
“Antibody”: This term includes, without distinguishing between different methods of genesis and formation, antibodies both against external antigens and against endogenous structures, i.e. autoantibodies, where the latter may also have become autoantibodies by antigen cross-reactions from antibodies against external antigens and may have preserved their binding capability with respect to external antigens.
   When, for example, it is stated that an antibody binds “to ganglioside structures and to antigen structures simulating ganglioside structures” or is “reactive towards gangliosides or certain gangliosides”, where reactive means “reactive in the context of specific binding”, it should be sufficiently defined by this definition without, for example, its specific binding also to additional other antigen structures, or its practical determination using reagents (for immobilization or marking or as competitors) with molecular structures which only simulate AG_{M1}, in particular the carbohydrate structure thereof, playing a role for the definition as antibodies according to the invention.
“Ganglioside” In the context of the present invention, the term “ganglioside” primarily represents the gangliosides AG_{M1} in the characterization of the binding behaviour of the antibodies to be determined. However, the term is also intended to include related gangliosides not investigated to date, for example fucosylated gangliosides, if it is found that antibodies also binding to these gangliosides and having a comparable diagnostic significance for neoplasms are found in cancer sera.
“Simulation” When, in the context of the present Application, it is stated that, in addition to the specific gangliosides (AG_{M1} and G_{M1}), substances or compounds “simulating” effects thereof may also be used, what is meant by this is that there are compounds which have carbohydrate structures (including bacterial toxins) and which bind to the antibodies in question like said gangliosides. They can therefore be potentially used, like the gangliosides themselves, for specific binding of the antibodies (for example for the purpose of removing them from the blood circulation) or for their blocking.
   In the stated context, substances “simulating” the binding behaviour of gangliosides can be found, for example, with the aid of a screening method in which a biological sample (in particular a serum) which has a high ganglioside antibody titre and whose binding behaviour was determined in a given assay is brought into contact with a candidate substance to be tested and the antibody binding is determined again in the same assay. A substantial reduction or elimination of the antibody binding is an indication that the substance investigated is a “ganglioside-simulating substance”. This test can also be carried out in association with establishing a patent infringement by agents which correspond to those disclosed or claimed in the present Application.

Further meanings of the terms are evident to a person skilled in the art from the introductory and following description of the invention and its embodiments.

In the description below, in addition to the content of a table, reference is also made to figures, which show the following:

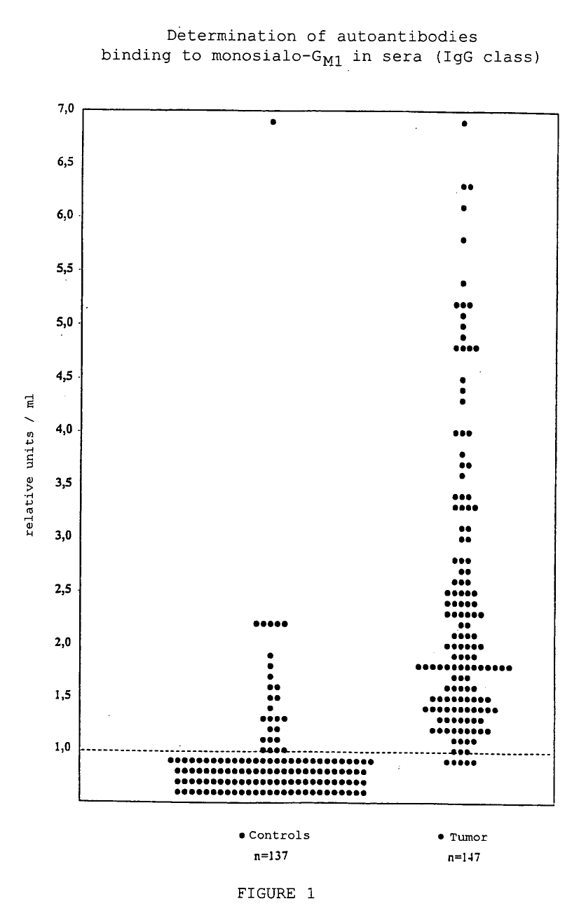

FIG. 1 shows a graph of the results of the measurement of antibodies of the IgG class which bind to monosialo-G_{M1}, in sera of 137 control persons, compared with the results of the measurement of sera of 147 tumour patients;

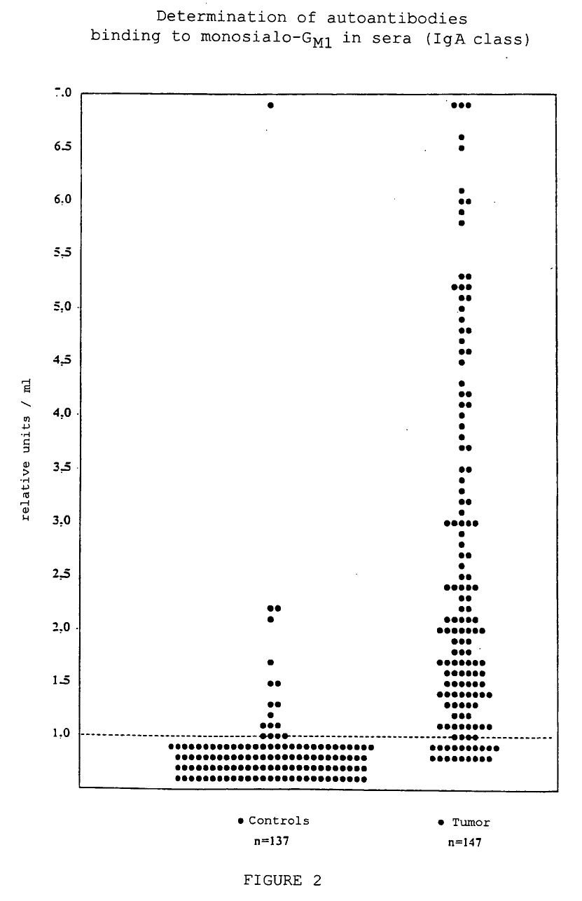

FIG. 2 shows the results of a measurement of the same sera as in FIG. 1 for antibodies of the IgA class which bind to monosialo-G_{M1};

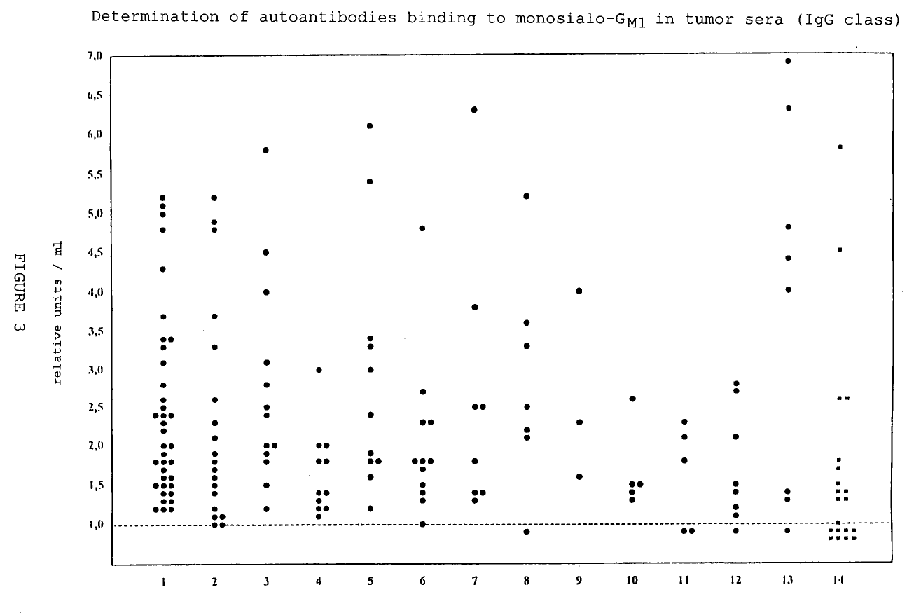

FIG. 3 shows a diagram in which there is a classification of the antibody determination in the tumour sera according to FIG. 1 with respect to the clinically diagnosed tumour types/diseases, which are characterized by numbers from 1 to 14, the meaning of the numbers 1 to 14 being explained in the description below;

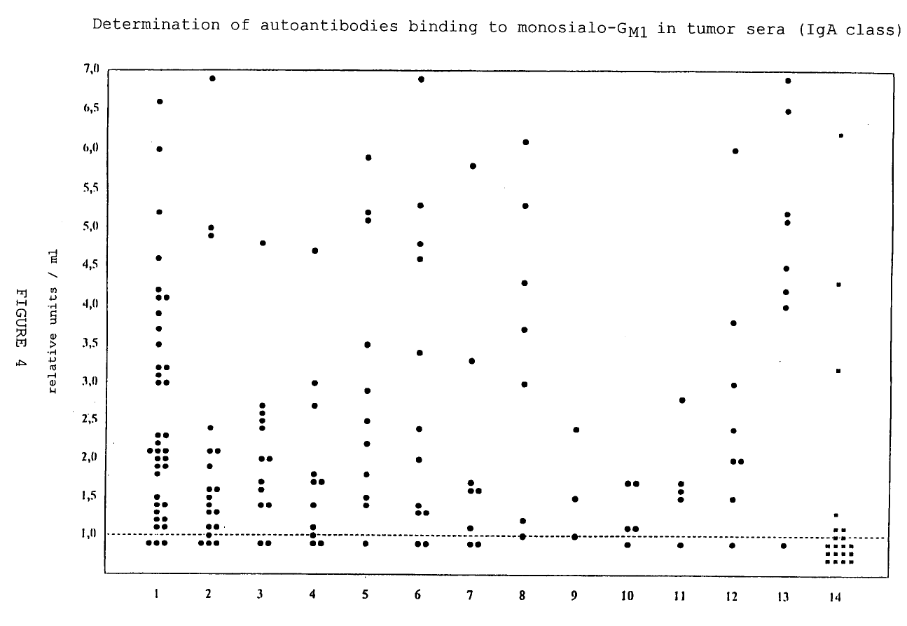

FIG. 4 shows a classification of the measurement of antibodies of the IgA class according to FIG. 2, corresponding to the classification of FIG. 3.

For antibodies which bind to asialo-G_{M1} (AG_{M1}) and are particularly important in the context of the present invention, findings corresponding to FIGS. 1 to 4 are available for a somewhat smaller group of sera. Reference is made to the corresponding experimental results in the simultaneously filed further patent application of the Applicant.

Below, the surprising analytical findings on which the present invention is based are first explained by way of example. A more detailed description is to be found in the simultaneously filed further application of the Applicant, which is hereby incorporated by reference for supplementing the disclosure of the present Application.

1. Measurement of Sera of Healthy Normal Persons (Controls) and Cancer Patients for (Auto)Antibodies Binding to the Gangliosides G_{M1} and AG_{M1}

Using test tubes coated with gangliosides (G_{M1} and AG_{M1}) (GA-CTs), the free binding sites of which had been saturated with BSA, series measurements were carried out on control sera and test sera. As described in more detail in the simultaneously filed further application of the Applicant, the content of which is hereby incorporated by reference for supplementing the content of the present Application, antibodies from the respective serum (control serum and test serum) were bound, in a first incubation step, to the gangliosides used for the coating, and then, for separate determination of antibodies of the IgG and IgA type, the bound antibodies were detected using marked animal (goat) anti-human IgG or anti-human IgA immunoglobulins. The bound marked immunoglobulins were detected and quantified on the basis of their marking (acridinium ester as chemiluminescence marker).

In order to obtain a measured signal representative of the amount of antibody, it is necessary to subtract a background signal which was obtained under identical measuring conditions for the respective identical serum using test tubes which, apart from the lacking ganglioside coating, were identical.

137 control sera (blood donor sera and—for avoiding age-related influences on the antibody concentrations—sera of normal persons of different ages from old people's homes and of the Applicant's employees) served as control sera for the antibody assays using GA-CTs which were coated with G_{M1}. For the antibody assays using GA-CTs which were coated with AG_{M1}, a partial group of these sera which comprised only 30 sera was measured.

147 sera of patients with clinically diagnosed tumours of various organs/tissues and, additionally, 20 sera of patients with chronic inflammatory intestinal diseases (Crohn's disease, ulcerative colitis) served as test sera for the antibody assays using GA-CTs which were coated with G_{M1}. For each test serum, there existed exact clinical documentation which permitted classification of the cancer patients' sera used in the measurement according to the type of tumour found in them. For the antibody assays using GA-CTs which were coated with AG_{M1}, a partial group of these sera which comprised only 30 sera of cancer patients was measured.

The results of the determinations of antibodies of the IgG and IgA classes using GA-CTs which were coated with G_{M1} are shown by way of example in FIGS. 1 and 2. More detailed measured data are to be found in turn in the Applicant's parallel Application of the same date.

FIGS. 3 and 4 show a classification of the measured results according to FIG. 1 (IgG) and FIG. 2 (IgA), respectively, according to clinical pictures. The index numbers plotted in the horizontal direction represent the clinical diagnoses shown in the following list:

| Index number | Diagnosis | Number of sera |
|---|---|---|
| 1 | Carcinoma of the colon | n = 37 |
| 2 | Carcinoma of the breast | m = 19 |
| 3 | Ovarian carcinoma | n = 13 |
| 4 | Carcinoma of the stomach | n = 11 |
| 5 | Carcinoma of the pancreas | n = 11 |
| 6 | Carcinoma of the oesophagus | n = 12 |
| 7 | Carcinoma of the gall | n = 8 |
| | bladder | |
| 8 | Liver carcinoma | n = 7 |
| 9 | C cell carcinoma | n = 3 |
| 10 | Thyroid carcinoma | n = 5 |
| 11 | Carcinoma of the prostate | n = 5 |
| 12 | Carcinoma of the lung | n = 8 |
| 13 | Other carcinomas | n = 8 |
| 14 | Crohn's disease/ulcerative | n = 20 |
| | colitis | |

The “other carcinomas” were distributed as follows:

Carcinoma of the appendix (n=1), carcinoma of the bladder (n=1), cardia carcinoma (n=1), distal verophagus carcinoma (n=2), carcinoma of the floor of the mouth (n=1), carcinoma of the kidney (n=1), epigastric tumour (n=1).

Table 1 below summarizes the results of FIGS. 3 and 4 numerically.

TABLE 1

| Organ | | | |
|---|---|---|---|
| affected | anti-GM₁ IgG | anti-G_{M1} IgA | n |
| Colon/Rectum | 38 = 100% | 35 = 92% | 38 |
| Breast | 19 = 100% | 16 = 84% | 19 |
| Ovaries | 13 = 100% | 11 = 85% | 13 |
| Stomach | 11 = 100% | 9 = 82% | 11 |
| Pancreas | 11 = 100% | 10 = 91% | 11 |
| Oesophagus | 12 = 100% | 10 = 83% | 12 |
| Gall bladder | 8 = 100% | 6 = 75% | 8 |
| Liver | 6 = 86% | 7 = 100% | 7 |
| C cell | 3 = 100% | 3 = 100% | 3 |
| Lung/Bronchia | 7 = 88% | 7 = 88% | 8 |
| Thyroid | 5 = 100% | 4 = 80% | 5 |
| Prostate | 3 = 60% | 4 = 80% | 6 |
| Other | 7 = 88% | 7 = 88% | 8 |

If a quantitative correlation of the measured results (not given in detail in this Application) obtained on the one hand with G_{M1}-coated test tubes and, on the other hand, for the same partial group of sera, with AG_{M1}-coated test tubes is additionally carried out for the IgG determination, there is substantial agreement which permits the conclusion that, in both determinations, at least very substantially identical or cross-reacting antibodies were measured, i.e. antibodies which have the reactivity of anti-AG_{M1} antibodies.

2. Discussion of the Findings of the Determination of Anti-Ganglioside Antibodies in Control Sera and in Sera of Cancer Patients

As impressively shown by the measured results summarized by way of example in FIGS. 1 to 4 and the above table 1, the determination of antibodies which bind to gangliosides (AG_{M1} and/or G_{M1}) permits a clear distinction between control sera and sera of cancer patients. Sensitivity of the determinations carried out was over 75% for all individual tumour types, and even 100% in most cases.

It should also be pointed out that, in an experiment corresponding to the determinations of antibodies of the IgG and IgA type and also intended to determine those of the IgM type, no diagnostically relevant results were obtained (results not shown).

The high sensitivities measured in combination with a high unspecificity with respect to the various cancer types make the determination of anti-ganglioside antibodies, in particular of those of the IgG and/or IgA classes, on the one hand, a promising assay method for the diagnosis, in particular for the early diagnosis, of neoplasms. The significantly increased titres of antibodies which bind to AG_{M1} or G_{M1} in all cancer sera, in particular sera of patients with a very wide range of cancer diseases, indicate that these are not increased in the context of an epiphenomenon but most probably play an important role in the context of establishing malignant neoplasms (tumours), which makes them therapeutic targets of considerable medical interest.

The scientific literature does not reveal any discoveries which refer to an important role of antibodies binding to gangliosides (in particular to AG_{M1} and therefore to G_{M1} with cross-reaction) in the genesis of cancer. There are only a few known papers in which an attempt was made also to determine anti-ganglioside antibodies in association with cancer diseases. Starting from the finding that an increased/abnormal expression of gangliosides was found in so-called “small cell lung cancer” (SCLC) in lung tissue of those suffering from the disease, two papers (Grazyna Adler et al., Small cell lung cancer is not associated with the presence of anti-fucosyl-GM1 ganglioside autoantibodies reactive in immunoenzymatic test, Lung Cancer 34 (2001) 383-385; Aleksandra Lewartowska et al., Ganglioside reactive antibodies of IgG and IgM class in sera of patients with differentiated thyroid cancer, Immunol. Lett. 80 (2002) 129-132), investigated whether this abnormal ganglioside expression means that anti-ganglioside antibodies can be formed and can be detected in cancer patients. In the first-mentioned paper, the primarily sought anti-fucosyl-GM1 ganglioside antibody was not found, whereas, in the second of the papers, small amounts of various anti-ganglioside antibodies were detectable for the case of differentiated thyroid cancer (DTC) in a certain number of patients, but, for example in the case of the anti-GM1 antibody assay, the values found for the cancer patients were below those of the controls (FIG. 1). Antibody titres which were increased to a slightly greater extent were found only with respect to binding to the fucosylated ganglioside FucGMl. In the light of the Applicant's findings, which clearly contradict those of the above papers, it is to be assumed that, in view of the considerable practical difficulties in the determination of anti-ganglioside antibodies against an intense, serum-dependent background signal, the authors were not able actually to produce informative measured results by means of the ELISA assay used by them. A possible pathogenic role of the antibodies which were to be determined is not suggested.

In a paper by Manousos M. Konstandoulakis et al., Autoantibodies in the Serum of Patients with Gastric Cancer: Their Prognostic Importance; Hybridoma, Vol. 17, No. 5, 1998, 431-435—continuing with similar papers by the same group of authors, in which autoantibodies known per se were determined in cancer patients—a determination of various types of antibodies in sera of patients suffering from stomach cancer is furthermore reported, and a possible prognostic role of such a determination is discussed. “Prognostic” is used there in the context of a prognosis for the further clinical course of an existing and diagnosed cancer disease. An ELISA assay is used for the antibody determination. The antibodies which were found at an increased level compared with controls also include so-called anti-G_{M1} antibodies, which however were found only at a sensitivity of about 35% in the patients suffering from the disease (compared with a value of 5% for normal persons). In the light of the stated paper and of other papers by the same authors, anti-ganglioside antibodies are only one of numerous types of autoantibodies investigated in connection with cancer, and no indication at all of a possible important role of such antibodies in the genesis and development of malignant neoplasms, and therefore also absolutely no promising therapeutic approach, can be derived from the reported data. A conclusion that the anti-G_{M1} antibodies determined might cross-react with the AG_{M1}-binding antibodies and might therefore have properties of such anti-AG_{M1} antibodies was not drawn in this context.

Such a perspective emerged for the first time from the measured data which were obtained by the Applicant using its considerable experience in the assay area and which were extremely surprising against the background of the prior art, namely the significantly increased occurrence, in the serum of all cancer patients, of antibodies binding to the ganglioside AG_{M1} and, cross-reacting therewith, also antibodies binding to G_{M1}.

On the basis of the novel findings of the measurements carried out by the Applicant, the Applicant was able to draw the conclusion that the increased antiganglioside (auto)antibody titres found in cancer patients are to be regarded as titres of antibodies inhibiting NK cells, with the result that these titres are of very novel serious medical importance which has nothing to do with the considerations which were the starting point for the papers discussed above, and that novel, promising therapeutic approaches can be derived from the found increased titres of anti-AG_{M1} antibodies and anti-G_{M1} antibodies cross-reacting therewith.

It is known that “natural killer cells” (NK cells; cytotoxically active lymphocytes) have, on their surface, asialo-G_{M1} structures to which anti-AG_{M1} antibodies can specifically bind, and that these subsequently deactivate and destroy the NK cells through their binding. However, active NK cells play an extremely important role in the human immune defence by, for example, killing all degenerated endogenous cells, e.g. cancer cells, which, owing to their degeneration, have lost the ability to inhibit the NK cells which come into contact with them. An impairment (inhibition, destruction) of the normal NK cells, which eliminates their selective cytotoxic properties, therefore leads to a situation where cells which have degenerated in the course of the natural life processes, in particular cells of the tumour cell type, are no longer properly eliminated. Owing to the improved survival prospects of such degenerated potential tumour cells, they can remain in the body when the NK cell function has been impaired, divide undisturbed and develop into an actual tumour. It should be pointed out that, in the area of animal experiments employing experimental animals in which an artificial cancer is induced, it is already customary to switch off the immune defence of the experimental animal by administration of anti-AG_{M1} antibodies in combination with a carcinogen or a tumour nucleus, so that the experimental cancer—desired in the animal model—can develop (cf. for example Hugh F. Pross et al., Role of Natural Killer Cells in Cancer, Nat Immun 1993; 12:279-292; Lewis L. Lanier et al., Arousal and inhibition of human NK Cells, Immunological Reviews 1997, Vol. 155:145-154; Yoichi Fuji et al., IgG Antibodies to AsialoGM1 Are More Sensitive than IgM Antibodies to Kill in vivo Natural Killer Cells and Prematured Cytotoxic T Lymphocytes of Mouse Spleen, Microbiol. Immunol. Vol. 34(6), 533-542, 1990; Carmine M. Volpe et al., AsGM1+NK Cells Prevent Metastatis of Invading LD-MCA-38 Tumor Cells in the Nude Mouse, J Surg Res 84, 157-161 (1999); Susan D Wilson et al., Correlation of Suppressed Natural Killer Cell Activity with Altered Host Resistance Models in B6C3F1 Mice, Toxicology and Applied Pharmacology 177, 208-218 (2001); H. Yoshino et al., Natural killer cell depletion by anti-asialo GM1 antiserum treatment enhances human hematopoietic stem cell engraftment in NOD/Shi-scid mice; Bone Marrow Transplantation (2000) 26, 1211-1216; N. Saijo et al., Analysis of Metastatic Spread and Growth of Tumor Cells in Mice with Depressed Natural Killer Activity by Anti-asialo GM1 Antibody or Anticancer Agents, J Cancer Res Clin Oncol (1984) 107: 157-163; Sonoku HABU et al., Role of Natural Killer Cells against Tumor growth in Nude Mice—A Brief Review, Tokai J Exp Clin Med., Vol. 8, No. 5, 6: 465-468, 1983; Lewis L. Lanier, NK Cell Receptors, Annu. Rev. Immunol. 1998, 16: 359-93; Theresa L. Whiteside et al., The role of natural killer cells in immune surveillance of cancer; Current Opinion in Immunology 1995, 7:704-710; Tuomo Timonen et al., Natural killer cell-target cell interactions, Current Opinion in Cell Biology 1997, 9:667-673).

Once the production of anti-G_{M1} antibodies has been initiated or greatly increased in a human individual, for example because of bacterial exposure (e.g. infections with Campylobacter jejuni or Helicobacter pylori), there is, taking into account the cross-reactivity of such antibodies with AG_{M1} epitopes which has been demonstrated by the Applicant (molecular mimicry), a precondition for potential damage to NK cells and hence the immune defence of the individual—resulting in an increased risk that degenerated tumour cells can develop into a tumour. It is therefore to be assumed that anti-AG_{M1} antibody titres found at significantly increased levels in all cancer sera which were measured in the above-mentioned assays are to a lesser extent a consequence of the presence of a neoplasm (tumour) in the sense of the known tumour markers, but rather a precondition or a promoting concomitant factor of its genesis. In the course of the tumour development, for example in combination with a stimulated NK selectivity, conversion of such antibodies into true autoantibodies and a “build-up” of the antibody titre may then occur in certain circumstances.

Since the antibodies cross-reacting with gangliosides and the effect on the immune system which is required for the production thereof may already be present before the development of a tumour, the determination of anti-AG_{M1} antibodies can be carried out for the purpose of establishing a disposition for the development of a cancer disease and, in the event of a positive detection of antibodies which bind to AG_{M1} and/or G_{M1}, it is possible to intervene in the context of cancer prevention and/or cancer inhibition or therapy.

In this connection, it is furthermore of considerable interest that, as shown in particular in FIGS. 3 and 4, increased titres of anti-G_{M1} antibodies cross-reacting with anti-AG_{M1} antibodies are also found in some patients who suffer from a chronic inflammatory intestinal disease (Crohn's disease, ulcerative colitis). It is known that, for example in the case of ulcerative colitis, there is an increased risk of the development of intestinal cancer in a later stage in the patients. The risk is about 40%. The finding that this percentage value is of the order of magnitude of the values which are found for increased anti-G_{M1} titres in sera of patients with chronic inflammatory intestinal diseases (index number 14), i.e. a parallelism of statistical cancer risk and the occurrence of anti-(A)G_{M1} antibodies is detectable in the sera of relevant patients, is a further finding supporting the above-mentioned assumptions.

The consequences for novel methods for the prevention, inhibition and therapy of cancer, which arise from the findings described, are as follows:

1. Measures for the intracorporeal blocking of the pathogenic antibodies by administering to the patient agents (which of course must have the required compatibility) which are suitable for saturating (“for blocking”) the antibodies binding to AG_{M1}. The gangliosides themselves are such agents, and their good compatibility for human patients is known from other contexts in which they have already been administered (they are administered in large amounts to patients suffering from Parkinson's disease).

2. Removal of the antibodies from the patient's circulation, for example, by extracorporeal binding to solid affinity materials (“plasmapheresis”). For the production of the affinity columns used as binders, it is also possible to use high-affinity carbohydrate structures which bind the antibodies with higher affinity than the gangliosides themselves.

3. Reduction of the endogenous production of the specific antibodies by administration of agents for blocking antigen-presenting cells or for producing T-cell anergy.

1. Use of the gangliosides asialo-G_{M1} (AG_{M1}) and/or G_{M1} and of substrates which simulate the carbohydrate moiety of these gangliosides with regard to binding to anti-AG_{M1} antibodies and/or anti-G_{M1} antibodies, for the preparation of an agent for binding or blocking anti-AG_{M1} antibodies and/or anti-G_{M1} antibodies which bind to the natural killer cells (NKC), for the prevention, inhibition and therapy of malignant cancer diseases.

2. Use of the gangliosides AG_{M1} and/or G_{M1} and of substrates which simulate the carbohydrate moiety of these gangliosides with regard to binding to anti-AG_{M1} antibodies and/or anti-G_{M1} antibodies, for the preparation of agents for cancer prevention, cancer inhibition and cancer therapy by blocking antigen-presenting cells or for producing T-cell anergy.

3. Use of the gangliosides AG_{M1} and/or G_{M1} and of substrates which simulate the carbohydrate moiety of these gangliosides with regard to binding to the anti-G_{M1} antibodies and/or anti-AG_{M1} antibodies, for the preparation of an affinity material for the extracorporeal removal of anti-AG_{M1} antibodies and/or anti-G_{M1} antibodies which bind to natural killer cells from the blood circulation of a patient.

4. Use according to claim 1, wherein the substance which simulate the carbohydrate moiety of these gangliosides with regard to binding to anti-AG_{M1} antibodies and/or anti-G_{M1} antibodies are characterized in that they are detectable with the aid of a screening method in which a substance to be tested is added to a serum having a high ganglioside antibody titre, the binding behaviour of the antibodies from the sample to the specific binding partners is then determined and a binding behaviour which is reduced compared with the substance-free sample is correlated with ganglioside simulation.

5. Use according to claim 2 for the preparation of an agent for blocking antigen-presenting cells or for producing t-cell anergy for administration by injection or for oral administration.

6. Use according to claim 1, wherein the cancer disease to be treated therapeutically or preventively with the agent is a carcinoma of the colon, a carcinoma of the breast, an ovarian carcinoma, a carcinoma of the stomach, a carcinoma of the pancreas, a carcinoma of the oesophagus, a carcinoma of the gall bladder, a liver carcinoma, a C cell carcinoma, a thyroid carcinoma, a carcinoma of the prostate, a carcinoma of the lung, a carcinoma of the appendix, a carcinoma of the bladder, a cardia carcinoma, a carcinoma of the distal verophagus, a carcinoma of the floor of the mouth, a carcinoma of the kidney or an epigastric carcinoma.

7. Use according to claim 1, wherein the patient is to be treated preventively or therapeutically with the agent is suffering from an inflammatory intestinal disease and, owing to the detection of significantly increased amounts of antibodies binding to the AG_{M1} and/or G_{M1}, has an increased risk of cancer of the intestine.

8. Agent for the prevention and treatment of cancer diseases, which, in addition to pharmaceutically acceptable excipients, contains active constituents which bind to AG_{M1} and/or G_{M1} antibodies in a manner such that the binding of these antibodies to natural killer cells (NKC) is completely or partly prevented.
